(19) United States
(12) Patent Application Publication
(10) Pub. No.: US 2023/0323267 A1
(43) Pub. Date: Oct. 12, 2023

(54) IN SITU CELL BIOREACTOR AND DELIVERY SYSTEM AND METHODS OF USING THE SAME

(71) Applicant: THE BOARD OF TRUSTEES OF THE UNIVERSITY OF ILLINOIS, Chicago, IL (US)

(72) Inventor: Ying LIU, Chicago, IL (US)

(21) Appl. No.: 18/043,136

(22) PCT Filed: Aug. 31, 2021
(86) PCT No.: PCT/US2021/048388

§ 371 Date: Feb. 27, 2023

Related U.S. Application Data
(60) Provisional application No. 63/073,382, filed on Sep. 1, 2020.

(51) Int. Cl. C12M 1/12; C12M 1/42; A61L 27/54

(52) U.S. Cl. CPC C12M 25/16; C12M 35/08; A61L 27/54; A61L 2400/06

(57) ABSTRACT

An in situ cell bioreactor and delivery system is provided. The system is composed of toroidal-spiral particles, which encapsulate cells therein and can further provide one or more active agents. Methods for using the system in cell-based therapies are also provided.

### INTRODUCTION

This application claims the benefit of priority from U.S. Provisional Application Ser. No. 63/073,382, filed Sep. 1, 2020, the content of which is incorporated herein by reference in its entirety.

### BACKGROUND

Cell-based therapies provide promising treatments for a number of pathologies including diabetes, Parkinson's disease, liver disease, heart disease, cancer and autoimmune disorders, to name a few. For example, adoptive cellular therapy (ACT) using ex vivo expanded patient tumor infiltrating lymphocytes (TIL) or genetically modified patient T cells has shown remarkable efficacies for treatments of lymphomas and leukemias, and is now also shedding light on certain types of solid tumors. T cells have also been genetically engineered with antitumor T cell receptors (TCRs) or chimeric antigen receptors (CARS) for treatment of a broader range of cancers. CAR T cell therapy has shown a remarkable effectiveness in patients with B cell malignancies such as chronic lymphocytic leukemia (CLL), acute lymphoblastic leukemia (ALL), acute myeloid leukemia (AML), diffusive large B-cell lymphoma (DLBCL), and follicular lymphoma. However, ACT is a “living” treatment because the administrated cells can proliferate in vivo, which makes the treatment more complicated in terms of dose design, delivery of co-stimulators or inhibitors, and safety evaluation. The antitumor functionality of ACT highly depends on the infused cells in vivo expansion and infiltration into the tumor microenvironment. Despite intensive studies, the success of ACT for treatment of solid tumors remains unsatisfactory; tumor regression is often transient, largely due to the poor persistence and functionality of transferred T cells, and immune suppression/exhaustion in the tumor microenvironment. One approach to boost the immune activities of ACT and reduce systematic toxicity has been to directly implant a cell-delivery device at or near the diseased site. For example, peptide-modified alginate-based discs, carrying cytotoxic T cells and porous micro silica particles loaded with interleukin 15 superagonist, have been shown to significantly enhance ACT efficacy in mouse breast and ovarian cancers (Stephan, et al. (2015) Nat. Biotechnol. 33(1):97-101).

Cell transplantation, using islets of Langerhans, stem cells, or β-cells, has also been shown to hold great promise as a therapeutic treatment for type 1 diabetes (T1D). Cell encapsulation technologies seek to address transplantation challenges via immunoisolation, safety, and clinical success. Microencapsulation, as one method for immunoisolation, has been intensively investigated using natural (e.g., alginate; Ibarra, et al. (2016) J. Biomed. Mater. Res. Part A 104:1581-1590) and synthetic hydrogel (e.g., PEG) beads (White, et al. (2020) ACS Biomater. Sci. Engineer. 6:2543-2562; Desai & Shea (2017) Nat. Rev. Drug Discov. 16:338-350; de Vos, et al. (2014) Adv. Drug Deliv. Rev. 67-68:15-34). Micro-sized capsules offer large surface area and allows for optimal passive transport of oxygen, nutrients, and insulin. However, due to the large islets quantities needed to reverse T1D, total retrieval and manipulation of transplanted capsules volume (˜10⁵ capsules) continues to be challenging, in particular for intraperitoneal (IP) implantation. Macro-encapsulation methods focus on the delivery of large amounts of islets within a single device. However, their relatively planar chambers tend to have lower surface area, reducing the metabolic efficacy of the device. In addition, large cell packing exacerbates cell oxygen competition, posing a major risk to graft functionality.

Accordingly, there is a need in the art for a safe, effective approach to transplant and optionally retrieve therapeutic cells for the treatment of diseases and conditions such as cancer, diabetes, Parkinson's disease, liver disease, heart disease, autoimmune disorders and the like.

### SUMMARY OF THE INVENTION

This invention provides an in situ cell bioreactor and delivery system composed of toroidal-spiral particles (TSPs), the TSPs including a biocompatible polymer matrix forming a channel and a compartment operatively connected to said channel, wherein said channel encapsulates cells (e.g., immune cells, chimeric antigen receptor T cells, T cells engineered to express modified T cell receptors, stem cells, or islets of Langerhans) suspended in a scaffold material that modulates activity and/or release of the cells from the toroidal-spiral particles and said compartment optionally includes one or more active agents. In some aspects, the biocompatible polymer matrix includes polyethylene glycol diacrylate, polyethylene glycol methacrylate, gelatin methacryloyl, methacrylated hyaluronic acid, methacrylated chitosan, methacrylated chondroitin sulfate, methacrylated glycol chitosan, methacrylated alginate, methacrylated dextran, methacrylated gellan gum, methacrylated poly(ε-caprolactone), dimethacrylate poly-D,L-lactide, dextran hydroxyl ethyl methacrylate, dextran mono(2-acryloyloxyethyl) succinate, polyglycerol-co-sebacate acrylate, α,ω-diacrylate polyethylene carbonate, polytrimethylene carbonate methacrylate, polyglycerol-co-sebacate-cinnamate, polyethylene glycol methyl ether methylacrylate, ethylene glycol dimethacrylate, polypropylene glycol methacrylate, or combinations or copolymers thereof. In other aspects, the scaffold material includes collagen, alginate, chitosan, fibrin, keratin, polyacrylamide, polyethylene glycol, hyaluronic acid, or a combination thereof or copolymer thereof. In further aspects, the one or more active agents can be immune checkpoint antibodies, therapeutic proteins or peptides, cancer chemotherapeutic drugs, immunoregulators, cytokines, vitamins, nutrients, diagnostic contrast agents, quantum dots, polymeric nanoparticles, or lipid nanoparticles and can optionally reside on a surface of the compartment, in the compartment, in the channel, or a combination thereof. In one particular aspect, the cells of the system are islets of Langerhans and the scaffold material is alginate. In another particular aspect, the cells of the system are immune cells or chimeric antigen receptor T cells, the scaffold material is collagen, and the one or more active agents include IL-2 and optionally a checkpoint antibody.

This invention also provides a method for producing the in situ cell bioreactor and delivery system by (a) preparing TSPs composed of a biocompatible polymer matrix forming a channel and a compartment operatively connected to said channel; (b) dehydrating the TSPs; and (c) introducing into the channel of the TSPs cells suspended in a scaffold material that modulates activity and/or release of the cells from the TSPs. In some aspects, the method further includes the step of introducing one or more active agents into the compartment of the TSPs prior to step (c). A kit for producing the in situ cell bioreactor and delivery system is also provided, which includes (a) dehydrated TSPs composed of a biocompatible polymer matrix forming a channel and a compartment operatively connected to said channel, wherein one or more active agents reside on a surface of the compartment, in the compartment, in the channel, or a combination thereof; and (b) a container including the scaffold material.

A method for delivering cells to a subject is also provides, which includes the step of administering to a subject in need of treatment with a cell therapy an effective amount of the in situ cell bioreactor and delivery system of the invention. In one particular aspect, the subject being treated has cancer such that the cells of the system are immune cells or chimeric antigen receptor T cells, the scaffold material is collagen, and the one or more active agents include IL-2 and optionally a checkpoint antibody. In another particular aspect, the subject has diabetes such that the cells of the system are islets of Langerhans and the scaffold material is alginate.

### BRIEF DESCRIPTION OF THE DRAWINGS

FIGS. 1A and 1B illustrate the morphology and usage of the toroidal-spiral particles (TSPs) for therapeutic cell encapsulation. FIG. 1A illustrates a TSP for T cell encapsulation, activation, expansion and delivery. I, IL-2; C, collagen scaffold material; T, T cell; M, biocompatible polymer matrix. FIG. 1B illustrates a TSP for islet of Langerhans encapsulation and implantation. TSPs prevent immune response and allow insulin and small molecule transport. I, islet of Langerhans; A, alginate scaffold material; M, biocompatible polymer matrix.

FIG. 2 shows the calculated diffusivity of insulin in PEG films made of 700-8k and 700-20k. The error bar is calculated as the standard deviation of n=3 samples.

FIG. 3 shows in vitro functionality of encapsulated human islets in TSPs made of blends of PEGDA of two molecular weights. Shown is the stimulation index (SI) from sets of naked islets and TSP encapsulated islets after incubation in Krebs-Ringer buffer (1 mL) with glucose. The error bars represent the standard deviation of n=3 groups.

FIG. 4 shows blood glucose levels (BGL) and body weight of diabetic mice before and after IP implantation of the TSP-encapsulated human islets. The TSPs were made of PEG 700-20k. The implantation was conducted on day 0, which is indicated by the dotted line. The solid line indicates normoglycemia at 220 mg/dL. Error bars correspond to standard deviation and significance as *p<0.05.

FIGS. 5A and 5B show islet functionality before and after the transplantation, respectively. FIG. 5A, shows insulin released and glucose stimulation index (SI) of the islets encapsulated in TSP (700-20k) prior to transplant (IP). The insulin released was normalized by islet equivalent (IEQ). FIG. 5B shows comparison of insulin released and glucose SI of retrieved TSPs from diabetic mice (TSP-D1, TSP-D2) and healthy mice (TSP-H) as compared to TSPs before transplantation (TSP-PreX). From each diabetic mouse, 3 sets of TSPs were collected to perform the GSIS (n=3). From 3 healthy mice, TSPs were collected and pooled into two sets (n=2) to perform the GSIS test.

FIG. 6 shows the proliferation rate of CD4 and CD8 T cells released from TSPs containing 0, 200 or 400 ng/particle IL-2.

FIG. 7 shows tumor growth curves for breast cancer (HCC1806 cells), pancreatic cancer (CanPan2 cells), and colorectal carcinoma (HCT116 cells) cells in NSG mouse models treated with phosphate-buffered saline (PBS), anti-mesothelin CAR T cells (i.v. or p.t. CAR T) or anti-mesothelin CAR T cells encapsulated in TSPs.

### DETAILED DESCRIPTION OF THE INVENTION

An in situ cell bioreactor and delivery system has now been developed that provides a well-controlled microenvironment for in situ cell proliferation, functionality, activation, expansion and release. The system uses heterogenous particles with toroidal-spiral internal structures, which are generated by a self-assembly process of polymeric droplet sedimentation in a miscible solution and subsequent polymer solidification initiated by photo cross-linking. These TSPs have been shown for use in the co-delivery of a small molecule drug and antibody (Sharma, et al. (2014) Biomacromolecules 15(3):756-762). In accordance with this invention, the large internal toroidal-spiral channel of such particles is filled with a scaffold material or biomatrix that functions as a cell incubator, allowing cell expansion and directed motion toward the opening of the TSP, while the outer main biocompatible polymer matrix provides mechanical strength and can be used to continuously supply nutrients, vitamins, signalling factors and the like to the cell reservoir. Using this cell delivery system, it has been shown that islets of Langerhans encapsulated in TSPs exhibit glucose-stimulated insulin secretion and excellent cell viability. In addition, there are minimal inflammatory responses after 4 weeks when the TSPs are transplanted into diabetic mice, and diabetic mice receiving said TSPs exhibit consistent hyperglycemic levels and stable blood glucose levels. Further, TSPs were shown to support the activation, proliferation and delivery of T cells in mice with a minimal inflammatory response after 10 weeks. Notably, T cells expressing an anti-mesothelin chimeric antigen receptor (CAR) effectively reduced tumor cell proliferation when implanted in immunodeficient mice bearing breast cancer, pancreatic cancer, or colorectal carcinoma tumor cells. Advantageously, by modifying the biocompatible polymer matrix and scaffold material, the cell delivery system of this invention can be tailored for the encapsulation, proliferation, and delivery of various cell types and sizes with predetermined rates of cell release as well as the controlled release of cell growth factors and/or therapeutic agents.

As indicated, the in situ cell bioreactor and delivery system of this invention is composed of polymeric toroidal-spiral particles or TSPs. TSPs are known in the art and described, e.g., in U.S. Pat. Nos. 8,852,645 and 9,974,839, incorporated herein by reference in their entireties. TSPs are prepared by taking advantage of the liquid-phase toroidal-spiral shape formed during sedimentation of a droplet of polymer solution (which may exhibit Newtonian or non-Newtonian rheology) through a miscible liquid of lower density. The TSP shape is then solidified by cross-linking the polymer to form a polymer matrix. While in some aspects the biocompatible polymer is chemically cross-linked (e.g., with divalent or trivalent cations), ideally the biocompatible polymer is photochemically cross-linked (e.g., with UV light).

For the purposes of this invention, the TSP is composed of a biocompatible polymer matrix, which protects the cells therein from the surrounding microenvironment (e.g., immune responses) and modulates activity and/or release of co-encapsulated active agents. A polymer matrix is “biocompatible” if it is generally non-toxic to cells or a recipient and does not cause any significant adverse effects to the subject. In some aspects, the biocompatible polymer matrix is also biodegradable. “Biodegradable” generally refers to a material that will degrade or erode by hydrolysis or enzymatic action under physiologic conditions to smaller units or chemical species that are capable of being metabolized, eliminated, or excreted by a subject. The degradation time is a function of polymer composition and morphology.

Representative examples of suitable biocompatible polymers of use in preparing the biocompatible polymer matrix include, but are not limited to, polyethylene glycol diacrylate (PEGMA), polyethylene glycol methacrylate (PEGMA), gelatin methacryloyl (GelMA), methacrylated hyaluronic acid (MeHA), methacrylated chitosan, methacrylated chondroitin sulfate, methacrylated glycol chitosan, methacrylated alginate, methacrylated dextran, methacrylated gellan gum, methacrylated poly(ε-caprolactone), dimethacrylate poly-D,L-lactide, dextran hydroxyl ethyl methacrylate (DEX-HEMA), dextran mono(2-acryloyloxyethyl) succinate (DEX-MAES), polyglycerol-co-sebacate acrylate (PGSA), α,ω-diacrylate polyethylene carbonate (PECDA), polytrimethylene carbonate methacrylate, polyglycerol-co-sebacate-cinnamate (PGS-CinA), polyethylene glycol methyl ether methylacrylate (PEGMEMA), polypropylene glycol methacrylate (PPGMA), ethylene glycol dimethacrylate (EGDMA), or combinations thereof or copolymers thereof (e.g., PEGMEMA-PPGMA-EGDMA copolymers). Notably, the selection and/or combination of the above-referenced polymers can be used to modulate mechanical strength, porosity, elasticity, rate of release of co-encapsulated active agents, and/or rate of degradation of the TSP. The selection of appropriate polymers for use in any particular application can be determined as described herein.

As with the biocompatible polymer matrix, the scaffold material in which the cells are suspended may be selected to modulate the release, e.g., the timing and/or rate, of the cells from the TSPs. A “scaffold material,” also referred to herein as a “biomatrix,” is a material or matrix that controls the migration (i.e., emigration) of resident cells or their progeny in time and space (directionally) through the TSP channel. The scaffold material may be a gel or semi-solid material in which the cells are suspended. Depending on the application for which the system is designed, the system can regulate emigration through the physical or chemical characteristics of the scaffold material itself. For example, if the permeability of the scaffold composition is adjusted, for example, by selecting or manipulating materials with respect to larger or smaller pore size, density, polymer cross-linking, stiffness, toughness, ductility, or viscoelasticity, cell mobility can be modulated. The scaffold material contains physical channels or passages through which cells can more easily move toward the opening of the channel of the TSP. In some aspects, the scaffold material is homogenous. Alternatively, the scaffold material may be organized into layers, each having a different permeability, so that the time required for the cells to move toward the channel opening is accurately and predictably controlled. Migration may also be regulated by scaffold composition degradation, dehydration or rehydration, oxidation, or chemical or pH changes. These processes are facilitated by diffusion or active motions of the cells controlled (e.g., binding, rolling, contraction, and relaxation), controlled by cellular detection of the gradient of enzymes, chemokines, or other reactive chemicals.

Preferably, the scaffold material has a predetermined rate of degradation based on temperature, pH, hydration state, and/or physical parameters such as porosity, cross-link density, type, and susceptibility to backbone chain chemistry or degradation. For example, calcium-crosslinked gels composed of high molecular weight high glucuronic acid alginate can take several months (1, 2, 4, 6, 8, 10, 12 months) to several years (1, 2, 5 years) in vivo to degrade. However, gels containing low molecular weight alginate and/or partially oxidized alginate will degrade over a period of weeks.

Exemplary scaffold materials include natural substances such as alginate, and alginate derivatives, chitosan, collagen, fibrin, hyaluronic acid, and keratin; synthetic materials such as polyacrylamide or polyethylene glycol; or combinations or copolymers thereof (e.g., polyethylene glycol-g-chitosan). Other suitable scaffold materials include polylactic acid, polyglycolic acid, poly(lactic-co-glycolic acid) polymers, gelatin, pullulan, scleroglucan, chitin, elsinan, xanthan gum, laminin rich gels, agarose, natural and synthetic polysaccharides, polyamino acids, polypeptides, polyester, polyanhydride, polyphosphadine, poly(vinyl alcohol), poly(alkylene oxide), poly(allylamine), poly acrylate), modified styrene polymer, pluronic polyol, polyoxamer, poly(uronic acid), polyvinyl pyrrolidone and any copolymer or graft copolymer described above. Preferred scaffold materials are collagen, fibrin, or alginate or alginate derivatives. Scaffold materials can be readily obtained from commercial sources such as BD Biosciences, Advanced BioMatrix, Baxter, Sigma, PRONOVA, BioTime Inc. and Corning.

To modulate migration, proliferation, viability, differentiation and/or activity of the cells or provide an additional therapeutic agent, one or more active agents can also optionally be included in the system. In some aspects, the one or more active agents reside on a surface of the compartment, in the compartment, and/or in the channel of the TSP. When located in the compartment of the TSP, active agents can move into the channel of the TSP, because the channel and compartment are operatively connected. As used herein, “operatively connected” means that the compartment of the TSP is in communication with the channel such that substances can move between the compartment and channel, e.g., by diffusion through the biocompatible polymer matrix.

In some aspects, the one or more active agents include immune checkpoint antibodies, therapeutic proteins or peptides, cancer chemotherapeutic agents, immunoregulators (e.g., immunosuppressives or immunostimulants), cytokines, vitamins, nutrients, diagnostic contrast agents, quantum dots, polymeric nanoparticles, or lipid nanoparticles. Suitable active agents of particular use in modulating migration, proliferation, viability, differentiation and/or activity of cells (internal and/or external of the TSP) generally include homing/migration factors, morphogens, differentiation factors, oligonucleotides, hormones, neurotransmitters, neurotransmitter or growth factor receptors, interferons, interleukins, chemokines, colony stimulating factors, chemotactic factors, extracellular matrix components, and adhesion molecules. Representative examples of active agents include parathyroid hormone (PTH), bone morphogenetic protein (BMP, e.g., BMP1, BMP2, BMP3, BMP4, BMP5, BMP6, BMP7, BMP8A, BMP8B), transforming growth factor-α (TGF-α), TGF-β1, TGF-β2, fibroblast growth factor (FGF), epidermal growth factor (EGF), platelet derived growth factor (PDGF), insulin-like growth factor (IGF), scatter factor/hepatocyte growth factor (HGF), fibrin, collagen (e.g., Col1a1, Col1a2, Col5a1, Col5a2, Col5a3, Col6a1, Col6a2, Col6a3, Col13a1), fibronectin, vitronectin, laminin, hyaluronic acid, an RGD-containing peptide or polypeptide, an angiopoietin and vascular endothelial cell growth factor (VEGF). Splice variants of any of the above-mentioned proteins, and small molecule agonists or antagonists thereof that may be used advantageously to alter the local balance of pro- and anti-migration and differentiation signals. Examples of cytokines as mentioned above include, but are not limited to interleukins (e.g., IL-1, IL-2, IL-4, IL-5, IL-6, IL-7, IL-10, IL-12, IL-15, IL-18), granulocyte-macrophage colony stimulating factor (GM-CSF), granulocyte colony stimulating factor (G-CSF), interferon-γ (IFN-γ), IFN-α, tumor necrosis factor (TNF), TGF-β, FLT-3 ligand, and CD40 ligand.

Therapeutic active agents such as immune checkpoint antibodies, therapeutic proteins or peptides, cancer chemotherapeutic agents, and immunoregulators can include, but are not limited to, DNA molecules, RNA molecules, antisense nucleic acids, ribozymes, plasmids, expression vectors, marker proteins, transcription or elongation factors, cell cycle control proteins, kinases, phosphatases, DNA repair proteins, oncogenes, tumor suppressors, angiogenic proteins, anti-angiogenic proteins, cell surface receptors, accessory signaling molecules, transport proteins, enzymes, anti-bacterial agents, anti-viral agents, antigens, immunogens, apoptosis-inducing agents, anti-apoptosis agents, and cytotoxins.

The amount of the one or more active agents in the TSP can be controlled by initial doping levels or concentration gradient of the substance, by embedding the active agents in scaffold material with a known leaching rate, by release as the scaffold material degrades, by diffusion through the biocompatible polymer matrix, by interaction of precursor chemicals diffusing into an area, or by production/secretion of compositions by resident support cells.

Cells that can be encapsulated within the cell bioreactor and delivery system of this invention include purified populations of cells or characterized mixtures of cells. Exemplary cells include, but are not limited to, various stem cell populations (embryonic stem cells differentiated into various cell types), bone marrow or adipose tissue-derived adult stem cells, mesenchymal stem cells, cardiac stem cells, pancreatic stem cells, endothelial progenitor cells, outgrowth endothelial cells, dendritic cells, hematopoietic stem cells, neural stem cells, satellite cells, and side population cells. Such cells may further include, but are not limited to, differentiated cell populations including immune cells (e.g., macrophages, T cells, B cells, NK cells, and dendritic cells), osteoprogenitors and osteoblasts, chondrocytes, keratinocytes for skin, tenocytes for tendon, intestinal epithelial cells, smooth muscle cells, cardiac muscle cells, epithelial cells, endothelial cells, urothelial cells, fibroblasts, myoblasts, chondroblasts, osteoclasts, hepatocytes, bile duct cells, islets of Langerhans, thyroid, parathyroid, adrenal, hypothalamic, pituitary, ovarian, testicular, salivary gland cells, adipocytes, and precursor cells. The cells of this invention may be autologous or heterologous, e.g., allogeneic. “Autologous” refers to a transplanted biological substance taken from the same individual. “Allogeneic” refers to a transplanted biological substance taken from a different individual of the same species.

Cells can optionally be genetically manipulated by the introduction of exogenous genetic sequences or the inactivation or modification of endogenous sequences. For example, T cells can be engineered to express modified T cell receptors (TCRs) or protein-fusion-derived chimeric antigen receptors (CARs) that have enhanced antigen specificity to provide a cell-based immunotherapy for the treatment of cancer. As a further example, recombinant genes are introduced to cause the cells to make proteins that are otherwise lacking in the host or target tissue. Production of scarce but desirable proteins (in the context of certain tissues) is augmented by transplanting genetically engineered cells. Cells used to seed the scaffold material may be capable of degrading the scaffold material over a desired period time in order to migrate through and out of the scaffold material. Scaffold materials can be selected such that they are susceptible to degradation by certain cell types seeded within the scaffold material. For example, scaffold materials and cells are selected and designed such that all or some of the cells seeded within the scaffold require a certain desired period of time to degrade the scaffold sufficiently to migrate through it and reach the surrounding tissue. The delay in the release of the cells to the surrounding tissue is controlled by varying the composition of the scaffold, to allow optimal time to signal the cells to multiply, differentiate, or achieve various phenotypes. General mammalian cell culture techniques, cell lines, and cell culture systems are described in Doyle, et al. (eds.) Cell and Tissue Culture: Laboratory Procedures, Wiley, 1998.

In certain aspects, the cells encapsulated in the system of this invention are immune cells, chimeric antigen receptor T cells, T cells engineered to express modified T cell receptors, stem cells or islets of Langerhans. In a particular aspect of this invention the cells encapsulated in the system are islets of Langerhans and the scaffold material includes alginate. In other aspects of this invention the cells encapsulated in the system are immune cells or chimeric antigen receptor T cells, the scaffold material includes collagen, and the one or more active agents include IL-2 and optionally a checkpoint antibody (e.g., a monoclonal antibody that targets either PD-1 or PD-L1).

Also provided herein is a method of producing the in situ cell bioreactor and delivery system. The method involves preparing TSPs composed of a biocompatible polymer matrix forming a channel and a compartment operatively connected to said channel; dehydrating the TSPs; and introducing into the channel of the TSPs cells suspended in a scaffold material that modulates activity and/or release of the cells from the TSPs. Polymeric TSPs within the scope of this invention are prepared by polymeric droplet sedimentation in a miscible solution and subsequent polymer solidification initiated by photo crosslinking. See, e.g., in U.S. Pat. Nos. 8,852,645 and 9,974,839, incorporated herein by reference in their entireties. Dehydration of the TSPs can also be achieved by placing the particles in sequentially increasing concentrations of ethanol, desiccation under vacuum, freeze-drying, air-convection or a combination thereof. Ideally, once the cells suspended in the scaffold material are introduced into the channel of the TSP, the cells are drawn into the channel by capillary force. In some aspects of the method, one or more active agents are loaded into the TSP prior to introducing the cells in the channel of the TSP. In accordance with this aspect, the dehydrated TSPs are contacted with a solution containing the one or more active agents and the one or more active agents are drawn into the compartment of the TSP by capillary force. Once loaded with the active agents, the TSPs are again dehydrated and subsequently loaded with the cells.

A kit for preparing the delivery system is also provided, which includes dehydrated toroidal-spiral particles composed of a biocompatible polymer matrix forming a channel and a compartment operatively connected to said channel, wherein one or more active agents reside on a surface of the compartment, in the compartment, in the channel, or a combination thereof; and a container including a scaffold material that modulates activity and/or release of the cells from the toroidal-spiral particles. The kit may further include instructions and/or reagents for isolating cells, as well as instructions for using the in situ cell bioreactor and delivery system. Kits suitable for a particular cell type and/or treatment of a particular disease or condition can be prepared. For example, a kit for preparing an in situ cell bioreactor and delivery system suitable for the treatment of a solid tumor may include a vial containing TSPs including IL-2 as an active agent and optionally a checkpoint antibody, a vial containing collagen as the scaffold material, and a vial containing a vector encoding a CAR that recognizes human mesothelin (e.g., MSLN-scFv-BEAM-CD28-4-1BB-CD3zeta).

Advantageously, the in situ cell bioreactor and delivery system of this invention not only provides a means for cells to be readily implanted into a subject, the cells in the TSPs can are protected from the host immune system such that they can proliferate, differentiate, perform specific cellular functions, migrate, or secrete biologically relevant enzymes, hormones or substances (e.g., insulin). Notably, all of these processes are carried out in situ under physiologically relevant conditions. As such, the in situ cell bioreactor and delivery system of the invention is of particular use in cell-based therapies. Accordingly, this invention also provides a method of delivering cells to a subject in need of treatment with a cell therapy. As used herein, “subject” is meant an individual. Thus, subjects include, for example, domesticated animals, such as cats and dogs, livestock (e.g., cattle, horses, pigs, sheep, and goats), laboratory animals (e.g., mice, rabbits, rats, and guinea pigs) mammals, non-human mammals, primates, non-human primates, rodents, birds, reptiles, amphibians, fish, and any other animal. The subject is preferably a mammal such as a primate or a human.

Although not precluded, treating a disease or condition does not require that the disease, condition, or symptoms associated therewith be completely eliminated, including the treatment of acute or chronic signs, symptoms and/or malfunctions. “Treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. “Treatment” therefore also includes relapse prophylaxis or phase prophylaxis. The term “treat” and synonyms contemplate administering the system of the invention to an individual in need of such treatment. A treatment can be orientated symptomatically, for example, to suppress symptoms. Treatment can be carried out over a short period, be oriented over a medium term, or can be a long-term treatment, for example within the context of a maintenance therapy.

Subjects in need of treatment in accordance with this invention include those with a disease or condition who would benefit from the administration of cell therapy. The cell delivery system of this invention is designed and manufactured for a wide variety of injuries, diseases, conditions and cell therapies, and delivered at or near to the treatment location using surgical, endoscopic, endovascular, and other techniques. The cell delivery system degrades, resorbs or can be removed after the treatment is successfully completed or can remain in place permanently or semi-permanently. Cells are seeded ex vivo into the TSPs with autologous or allogeneic cells. The system is particularly useful in providing support to damaged or diseased tissue (e.g., providing enzymes or hormones such as insulin), providing immune cells (e.g., immunotherapy), regenerating dermal tissue (e.g., scarring, ulcers, burns) or central nervous system tissue (spinal cord injury, multiple sclerosis, amyotrophic lateral sclerosis, dopamine shortage), or for skeletal-muscle system repairs (tendons, ligaments, discs, post-surgical, hernias).

A method for treating a subject in need of cell therapy involves the step of administering or implanting the cell delivery system in or near the tissue in need of support, regeneration, repair, or replacement. The system is useful to treat acute and chronic tissue disease or defects in humans as well as animals such as dogs, cats, horses, and other domesticated and wild animals. Diseases or conditions treated include, but are not limited to, cancer, autoimmune diseases such as diabetes, lupus, mastocytosis, scleroderma, and rheumatoid arthritis. The system is also useful to supply cells for treating blood disorders such as sickle-cell anemia or vascular disorders such as peripheral arterial disease or peripheral ischemia. Neuropathological disorders such as Amyotrophic Lateral Sclerosis (ALS), polyneuropathy, multiple sclerosis (MS), Parkinson's disease, and epilepsy may also be treated, as can retinal diseases such as retinal degeneration and corneal injury (caustic) also can be treated with the system. The system can also be used to treat various heart and respiratory diseases such as myocardial infarction (MI), congestive heart failure (CHF), coronary artery disease (CAD), and cardiomyopathy or respiratory diseases, e.g., chronic respiratory diseases (CRDs) or pulmonary fibrosis, respectively. Additionally, the system can be used to treat bone and cartilage defects/diseases such as periodontitis, osteoarthritis, or a skull injury. Moreover, the system can be implanted into or adjacent to neural tissues, e.g., to treat spinal cord injuries such as a crushed spinal cord or to provide pain management. Other diseases the system can be used to treat are graft vs host disease, renal failure, Crohn's disease, skin diseases or injuries (e.g., burns and ulcers), surgical defects such as those resulting from Caesarian section births and those resulting from cosmetic surgery, or to prevent age-related alterations or deterioration.

In certain aspects, the method of delivering cells is to a subject diagnosed with cancer. In accordance with this aspect, the cells being administered are chimeric antigen receptor T cells, the one or more active agents include IL-2 and optionally a checkpoint antibody. In other aspects, the method of delivering cells is to a subject diagnosed with diabetes. In accordance with this aspect, the cells being administered are islets of Langerhans, and the scaffold material includes alginate.

The cell delivery system may be administered by a variety of routes, wherein the route selected will be dependent on the disease or condition to be treated. Exemplary routes of administration include, but are not limited to, subcutaneous, intraperitoneal, intratumor, intramuscular, intrabone, intracardiac, and the like. In certain embodiments, administration involves providing to a subject about 10², 10⁴, 10⁶, 10⁷, 10⁸, 10⁹, 10¹⁰, 10¹², or more cells. The number of cells administered may be chosen based on the route of administration and/or the condition for which the cells are administered. The delivery system of the invention can be formulated for administration to include the necessary physiologically acceptable carrier material, excipient, lubricant, buffer, surfactant, antibacterial, bulking agent (such as mannitol), antioxidants (ascorbic acid or sodium bisulfite), and the like.

The system of this invention can increase the efficacy of stem and transgenic cell therapies and can be tailored to suit each clinical problem with the appropriate choice of TSP composition, scaffold composition, active agent(s) and/or cell types. The system solves the problem of efficiently integrating therapeutic cells into target tissue. Physicians place the system near the site requiring therapy or regeneration, where it delivers a flow of cells to the target site. Unlike traditional compositions, the system exports cells after they have incubated, replicated and matured inside the system. The system has shown improvements in viable cell delivery and proliferation. The system can be configured to release the therapeutic cells while maintaining a viability rate of greater than 50% of the cells encapsulated therein. Thus, fewer cells are needed per treatment allowing successful therapies which might have failed at lower cell delivery rates. Lower cell numbers also permit autologous grafts, because fewer cells need to be harvested from the subject to be treated and less time is required between harvest and graft. Since fewer cells are required, relatively rare cells can be used. The system also permits less expensive allogeneic grafts. Other advantages include rapid determination of the therapeutic benefit of any treatment and faster tissue growth and enhanced healing.

The following non-limiting examples are provided to further illustrate the present invention.

### Example 1: Heterogeneous Polymeric Particles Encapsulating Human T Cells

Materials. Poly (ethylene glycol) diacrylate (PEGDA) with molecular weights of 700 (PEGDA 700) and 8000 (PEGDA 8000), low viscosity alginate (in the powder form of alginic acid sodium salt from brown algae), centrifugation medium sold under the tradename FICOLL-PAQUE® Plus (manufactured by GE Healthcare), fetal bovine serum, glycerol, and collagenase type 2 (manufactured by Worthington) were purchased from Sigma-Aldrich (St. Louis, MO). Photo initiator sold under the tradename IRGACURE® 2959 (2-hydroxy-1-[4-(2-hydroxyethoxy) phenyl]-2-methyl-1-propanone; I-2959) was from BASF (Florham Park, NJ). Bovine collagen (3 mg/ml) was purchased from Advanced BioMatrix (Carlsbad, CA). Recombinant human interleukin 2 (IL-2) was purchased from PeproTech (Rocky Hill, NJ). CTS™ OpTmizer™ T Cell Expansion medium, 1×Penicillin-Streptomycin, human IL-2 ELISA kit (produced by Invitrogen), L-glutamine (produced by Gibco), and AlgiMatrix™ dissolving buffer were purchased from ThermoFisher (Waltham, MA). Human antibodies CD3 and CD28 were purchased from BioXCell (West Lebanon, NH). Molecular grade water and 10×phosphate-buffered saline (PBS) were purchased from Corning (Corning, NY). NaOH was purchased from EM Science (Gibbstown, NJ).

Toroidal-Spiral Particle (TSP) Preparation. A polymeric drop containing PEGDA, water, ethanol, and photo initiator was infused into a bulk solution composed of glycerol and ethanol. When the droplet formed a desired shape during sedimentation through the stratified bulk solution, PEGDA cross-linking was initiated by high-intensity UV (˜10 W/cm², BlueWave 75, Dymax Corp) exposure and particle structure became solidified. Morphology of the particles can be manipulated by varying the fluid and flow conditions (Szymusiak, et al. (2012) Soft Matter. 8(29):7556-7559). More specifically, to generate the TSPs used in this study, the polymer droplet contained 24 wt % PEGDA (MW 700), 16 wt % PEGDA (MW 8000), 34 wt % glycerol, 18 wt % water, 7.97 wt % ethanol and 0.03 wt % photoinitiator I-2959; the bulk solution was composed of 56 wt % glycerol and 44 wt % ethanol. TSPs made of PEG700 were made from a polymer droplet containing 40 wt % PEGDA (MW 700). Particles were sequentially soaked in ethanol, 70% ethanol, 20% ethanol, and water for at least one day in each to be sterilized and remove residue photo initiator. Sterilized TSPs were stored in molecular grade water until used.

T Cell Culture and Activation. For human whole blood collected from different donors, Institutional Review Board (IRB) approval was obtained from the University of Illinois at Chicago, and informed consent from volunteers was obtained. Peripheral blood mononuclear cells (PBMCs) were isolated from the whole blood by standard leukapheresis using centrifugation medium sold under the tradename FICOLL-PAQUE® per the instruction of the manufacturer, and cultured in CTS™ OpTmizer™ T Cell Expansion medium supplemented with 10% FBS, 1× Penicillin-Streptomycin, 1× glutamine, and 30 U/ml recombinant human IL-2 in a 37° C., 5% CO₂ incubator. T cells were activated on the second or third day after PBMC isolation using immobilized anti-CD3 and soluble anti-CD28. More specifically, the following steps were sequentially taken to activate T cells: anti-CD3 solution (50 μL, 7.5 μg/ml) was added to each well on a 96-well plate and incubated for 2 hours in a 37° C., 5% CO₂ incubator or overnight in a 4° C. refrigerator to immobilize anti-CD3 on the well bottom; the antibody solution was then decanted and the wells were washed with PBS two times to eliminate excess anti-CD3; the cell suspension (200 μL, 7.5×10⁵ cells/ml) containing 1 μg/ml anti-CD28 was added into each well. Cell medium was supplemented with 100 U/ml IL-2 and replaced every 2 days. Dead cells were eliminated by using centrifugation medium and gradient centrifugation prior to loading cells into the TSPs.

IL-2 Encapsulation in TSPs. TSPs were dehydrated in a 37° C. oven for 15 minutes. The dehydrated TSPs were stationed on a custom-made plate with the opening pointing upwards. A drop of 2 μL of IL-2 solution, with a concentration of 250 μg/ml, was added to the opening of the TSP by using a pipette. IL-2 solution was driven into the channel of the TSP by capillary force. After the IL-2 solution was completely absorbed, the TSP was dehydrated again and prepared for loading T cells.

T Cell Loading into TSPs. T cells were loaded into TSPs after 6 to 8 days of cell activation once a large enough cell number was reached. The activated T cells were suspended in a biomatrix composed of collagen, alginate, or an equal-volume mixture of the two polymers. Collagen was neutralized beforehand with 10×PBS and 0.1M NaOH. Specifically, a collagen solution was mixed with 10×PBS and water at an 8:1:1 ratio by volume, and 0.1M NaOH was added to adjust the pH to be in the range of 7.0-7.4. The cell suspension (10-12 μL) was added to the opening of the TSPs drop-wise using a pipette. When filling the toroidal-spiral channels of the particles with neutralized collagen, the infusion process was completed on ice to prevent pre-mature collagen solidification. After cells were loaded into the TSPs, collagen was cross-linked by temperature elevation during cell culturing in a 37° C. incubator. Alginate or an alginate-collagen mixture was crosslinked by immersing the TSPs in a 20 mM CaCl₂) solution for 5 minutes. The particles were washed to remove excess CaCl₂), transferred into the wells of a 96-well plate filled with cell medium, and placed into the incubator. About 3.3×10⁵ cells were loaded into each TSP for measurements of cell release.

Measurements of Cell Release from TSPs. TSPs were cultured on a 96-well plate with three TSPs in each well. The measurements started 2 days after the cells were loaded into the TSPs and repeated every two days. For each measurement, all the cell medium was collected from the well and fresh cell medium was replenished. Cell density was counted with a hemocytometer. The number of cells released was calculated by multiplying the cell density and the volume of the cell medium extracted.

Cell Viability. Fluorescein diacetate (FDA) and propidium iodide (PI) were used to stain the cells to indicate living (green) and dead (red) cells, respectively. TSPs at various days of cell culturing were immersed in 0.9 μM FDA and 28 μM PI in PBS and settled for 15 minutes to allow stains to fully diffuse into the toroidal-spiral channel. Fluorescence microscopy (Olympus IX70, Japan, equipped with a Tucsen ISH1000 camera (Tucsen Photonics)) was used to record the fluorescent images for cell viability analysis.

Visualization of T Cell Mobility in Biomatrices. T cell mobility in 3D biomatrices composed of alginate, collagen, or an equal-volume mixture of the two were visualized by an inverted microscope (Olympus IX73, Japan) with a CCD camera (Olympus XM10, Japan) with a 40× magnification lens for 30 minutes at a rate of 1 frame/second. Cell suspensions were mixed with an equal volume of the biomatrix in the wells of a 96-well plate to reach a final concentration of 1.5×10⁶ cells/ml and 100 μL total volume. More specifically, the compositions of the biomatrices were as follows: alginate biomatrix composed of 45 μL 2% alginate and 5 μL 50 mM CaCl₂); alginate-collagen mixture composed of 22.5 μL 2% alginate, 2.5 μL 50 mM CaCl₂) and 25 μL neutralized 3 mg/ml collagen; collagen biomatrix composed of 50 μL neutralized 3 mg/ml collagen. All groups were placed in a 37° C. incubator overnight to fully cross-link the biomatrices prior to tracking cell motions.

Videos were recorded at three randomized locations for each biomatrix. The films were analyzed using the TrackMate package of ImageJ to acquire the trajectories of cells (Tinevez, et al. (2017) Methods 115:80-90). Displacement was defined as the distance between the initial and final locations of the cell; travel distance was defined as the length traveled by a cell for the investigated time span. Average values and error bars were obtained from the statistical analysis of all the cells at the three locations.

Cell release from the TSPs was also visualized using the same camera with a 20× magnification lens. For video analysis, the TSP encapsulating 200 ng of IL-2 in the main polymer matrix and about 1 million cells in the channel were placed in an incubator overnight. Films were recorded by a CCD camera for 60 minutes at a rate of 1 frame/second.

Measurements of Cell Expansion. Cells suspensions were mixed with an equal volume of the biomatrix in the wells of a 96-well plate to reach a final concentration of 3.5×10⁵ cells/ml with 100 μL total volume. The method to trigger biomatrix cross-linking was the same as described in the aforementioned section of T Cell Mobility in Biomatrices. After 2 days of cell seeding, collagen and alginate were decomposed with 25 U/ml collagenase and AlgiMatrix™ dissolving buffer, respectively. Cell concentration was measured with a hemocytometer. The total number of cells was calculated by multiplying the measured cell concentration with the volume of the solution. Fold cell expansion was obtained by dividing the final cell counts by the original number of cells seeded in the well.

Measurements of IL-2 Release from TSPs. IL-2 was encapsulated in the main polymer matrix of the TSPs by adding 2 μL IL-2 solution to the toroidal-spiral channel of the dehydrated particle through the opening. The channel was then filled with the neutralized collagen. The TSPs were placed in the wells of a 96-well plate with two particles in each well. PBS buffer (200 μL) was added to each well and replaced every two days. The collected supernatant was placed in a −80° C. freezer until Sandwich Enzyme-Linked ImmunoSorbent Assay (ELISA) was performed. Absorbance was read on a UV-Vis plate reader (SpectraMax M2, Molecular Devices).

In vivo Biocompatibility Study. C57BL/6 Mice were anaesthetized with isoflurane and prepared for surgery by scrubbing with 70% alcohol. Three TSPs were inserted subcutaneously on the back through a 1-2 cm incision. At 2 and 10 weeks after implantation, mice were sacrificed and TSPs along with surrounding tissues were harvested. Samples were then fixed with 10% formaldehyde for 24 hours and stored in 70% alcohol. The fixed samples were embedded, sectioned into 5 μm and haematoxylin and eosin (H&E) stained following a standard protocol. Briefly, formalin-fixed tissue was processed on ASP300 S automated tissue processor (Leica Biosystems) using a standard overnight processing protocol. Tissue was embedded into paraffin blocks and sectioned at 5 μm. Tissue sections were baked, deparaffinized and stained with hematoxylin and eosin on Autostainer XL (Leica Biosystems) following a preset protocol.

Results. Poly (ethylene glycol) diacrylate (a mixture of two molecular weights, 700 and 8000, 60/40 v/v) (PEGDA 700/8000) hydrogels, a well-investigated biocompatible polymer, was selected as the material of the main polymer matrix to provide mechanical strength and elasticity for injection and sustained release of growth factors. PEG is considered a non-degradable polymer, because of its lack of hydrolytic and enzymatic degradable sites. Therefore, delivery systems made from PEG can provide long-term sustained release. To modify the degradation rate, different functional end groups can be added to PEG chains and biodegradable polymers can be blended with PEGDA to form the hydrogel. In this respect, TSPs were generated by mixing PEGDA with gelatin methacryloyl (GelMA), which can be degraded with the presence of matrix metalloproteinases. The degradation rates were tunable by varying the ratio of PEGDA and GelMA. The TSPs were sterilized by 70% ethanol and stored in DI water.

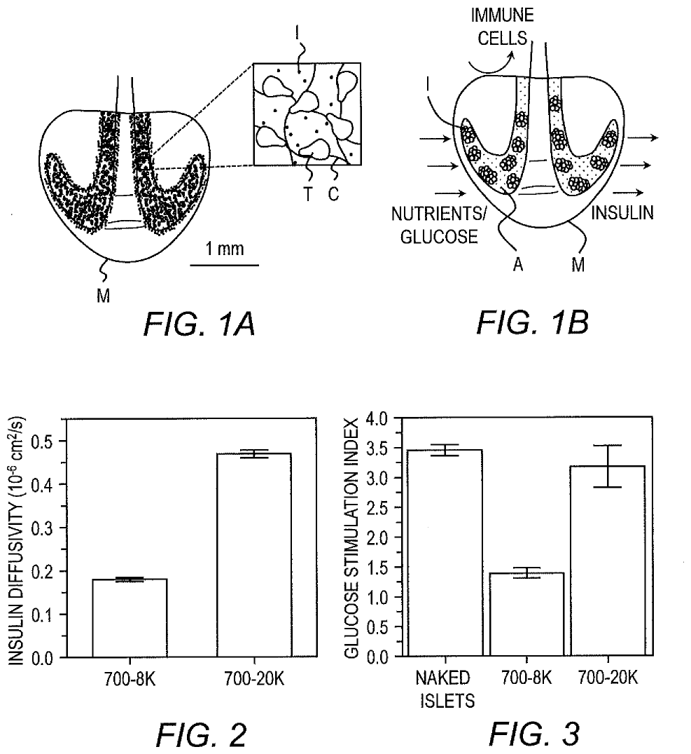

Prior to cell loading, the TSPs were dehydrated in a vacuum oven. A wide range of small or macro molecule compounds can be loaded into the main polymer matrix of the TSPs through entrainment of the compounds dissolved in the bulk solution, droplet interaction during TSP formation, or polymer rehydration after particle formation (Sharma, et al. (2012) Langmuir 28(1):729-35; Sharma, et al. (2014) Biomacromolecules 15(3):756-762). In this study, interleukin 2 (IL-2) was encapsulated in the main polymer matrix, close to the internal surface of the toroidal-spiral channel through polymer rehydration, to support the proliferation of the T cells. More specifically, IL-2 solution (with a concentration of 250 μg/ml IL-2 in DI water) was added to the opening of the channel, which was driven into the toroidal-spiral channel and then deposited in a thin layer on the main polymer matrix by the capillary force during polymer rehydration. Human T cells, harvested from activated PBMC using immobilized anti-CD3 and soluble anti-CD28, were suspended in a biocompatible matrix (e.g., collagen), and subsequently loaded into the vacant internal channel of the TSPs by a similar process of polymer rehydration (FIG. 1A).

The mobility and release of cells is highly dependent on the biomatrix used as it provides a guidance structure for cell directional migration, promotes cell contact and communication to seed cell proliferation, and presents a physical barrier by forming heterogeneous networks with subcellular-size pores (Huang, et al. (2017) Chem. Rev. 117(20):12764-12850). Therefore, the efficiency and direction of in vitro cell migration can be manipulated by tuning the structural components of the biomatrix in the TSP channel. Two types of biopolymers commonly used for cell encapsulation, collagen and alginate, were tested. Collagen is the most abundant extracellular matrix (ECM). Many sources of collagen are available to modulate cell behavior. In the present case, bovine collagen was used to demonstrate the predictable cell mobility and release. By loading the biomatrix into the internal toroidal-spiral channel, mechanical strength of the biomatrix was no longer a concern. Alginate, because of its biocompatibility and structural support, has also been widely investigated for cell encapsulation for diabetes treatments, bone regeneration, and the like (de Vos, et al. (2014) Adv. Drug Deliver. Rev. 67-68:15-34; Van Vlierberghe, et al. (2011) Biomacromolecules 12(5):1387-1408). To enhance the cell migration in alginate, cell adhesion peptides can be conjugated to alginate to mimic the handles of the collagen (Stephan, et al. (2015) Nat. Biotechnol. 33(1):97-101).

Cell mobility and release rates of T cells suspended in collagen, alginate, or mixtures of the two were monitored subsequent to loading into TSPs. When the composition of the biomatrix transitioned from pure alginate to pure collagen, mobility of the T cells increased for both total travel distance and displacement as revealed by time-lapse microscopy measurements. This result is consistent with the expression of collagen-binding integrins (such as α1β1 and α2β1) on T cells, which promote cell attachment to and migration in collagen (Andreasen, et al. (2003) J. Immunol. 171(6):2804-11; Naci, et al. (2014) Cell Signal. 26(9):2008-15). The average speed of T cell migration in collagen was about 5.1±0.9 μm/min, close to the previously reported value (7.5±2.25 μm/min; Niggemann, et al. (1997) Cancer Lett. 118(2):173-180). The average migration speed of T cells in alginate was much slower, about 3.2±0.5 μm/min. In 30 minutes, T cells traveled 152.4 μm±28.3 μm in collagen, but only 96.3 μm±15.1 μm in alginate. The distance displacement of T cells in collagen in 30 minutes was 7.3 μm±3.3 μm, lower than the reported value (16.8±1.8 μm; Applegate, et al. (1990) Cancer Res. 50(22):7153-7158), which might be ascribed to the denser matrix network resulting from the higher concentration of collagen used in this study. However, varying the ratio of alginate to collagen from 1:1 to 1:2 had minimal effects on cell mobility and displacement. This may have been due to the nonlinear coupling effects of the density of the collagen binding receptors and the porosity of the matrix. The rate of cell proliferation in different biomatrices, a critical factor for determining cell release rate and sustainability, was also monitored. After the T cells were seeded in collagen, alginate, or the mixture of the two polymers with an initial culturing concentration of 0.5×10⁶ cells/ml, the number of T cells in collagen almost tripled in 2 days while no obvious increase was observed in the other two biomatrices. Binding with collagen receptors regulates cell proliferation and differentiation, which was apparently not achieved using alginate or the mixture of alginate and collagen.

The release rate of human T cells from the TSPs was measured under various conditions. About 0.33 million cells were loaded into each TSP. For each test, three particles were placed in 200 μL of release medium (CTS™ OpTmizer™ T Cell Expansion SFM, supplemented with 10% FBS, 1× Penicillin-Streptomycin, and 1× glutamine). The culture medium was replaced every two days and the cells in the medium were counted each time. The corresponding dependence of cell release rate on the biomatrix in the toroidal-spiral channel was tested. In this case, 100 U/ml IL-2 was supplemented in the culture medium. The rate of cell release doubled when collagen was increased from 50% (50% collagen/50% alginate) to 100% in the biomatrix. When collagen was used to suspend the cells, more than 2.5 million T cells were released from the TSPs over a month, which was more than 2.5 times the initial number of cells loaded in the particles. The number of cells released from the TSPs with pure alginate in the channel was below the detection limit of the hemocytometer. Cell release could be visualized at the opening of the toroidal-spiral channel when collagen; however, this event was harder to catch for cells suspended in alginate or the mixture of alginate and collagen. The higher release rate of the T cells from the TSPs filled with collagen compared to alginate was likely due to the faster cell migration and proliferation in collagen. These analyses collectively indicate that by tuning the composition of the biomatrix, cell release rates can be modulated.

An essential feature of the TSPs is co-encapsulation and sustained release of growth factors to support cell proliferation, activity and/or viability. In this study, interleukin 2 (IL-2) was either supplied in the culture medium or incorporated in the main polymer matrix near the internal surface of the toroidal-spiral channel. IL-2 is known to play a role in lymphocyte activation and proliferation. Given that the tumor microenvironment provides a limited amount of T cell growth factors and contains a significant number of immunosuppressive factors (Gajewski, et al. (2013) Curr. Opin. Immunol. 25(2):268-276; Blank, et al. (2004) Cancer Res. 64(3):1140-1145), cell release rates were also determined with TSPs suspended in a culture medium lacking an external supply of IL-2. With 500 ng IL-2 encapsulated in the main polymer matrix of the TSP, the cell release rate was very similar to that when supplying 100 U/ml (46 ng/ml) IL-2 in the culture medium, i.e., approximately 2.5×10⁶ cells released after 30 days. Without IL-2 preloaded in the TSP or in the culture medium, the number of cells released from the TSPs dropped after one week, with a total of less than 1 million cells released, an amount that was comparable to the number of cells originally loaded into the TSPs. Initial cell viability was found to be similar for T cells encapsulated in the TSPs regardless of the presence or absence of IL-2. However, the continuous availability of IL-2 for the activated T cells was essential for robust cell expansion and sustained cell release. Thus, in microenvironments (e.g., the tumor microenvironment) having a limited supply of IL-2, the heterogeneous structure of the TSPs offers an engineered approach to provide IL-2 to the cells for cell expansion.

Despite its essential role in cell activation and proliferation, levels of IL-2 release must be controlled, since excess amounts of IL-2 can cause serious side effects, such as respiratory distress and arrhythmias (White, et al. (1994) Cancer 74(12):3212-22). The release of IL-2 from the polymer matrix is dominated by molecular diffusion driven by concentration gradients. Because the main polymer matrix of PEGDA 700/8000 is dense with small pores and low porosity and IL-2 is primarily localized at the thin layer close to the toroidal-spiral channels, the release of IL-2 can be deemed a two-step process: (1) detachment from the surface of the toroidal-spiral channel or diffusing from the main polymer matrix of PEG to collagen, and (2) revolving through the long toroidal-spiral channel to the PBS buffer at the channel opening. The rate of IL-2 release from the TSPs into the PBS buffer was measured for three initial loading amounts of IL-2 (150 ng, 600 ng and 1500 ng). For all three groups, only a small amount (less than 1%) of IL-2 was accumulatively released into the PBS buffer in two weeks. The diffusion time in the toroidal-spiral channel filled with collagen can be estimated by l²/D, where D is the diffusivity of IL-2 in collagen and l is the revolving distance from the opening. Using the human IL-2 diffusivity in gelatin hydrogel, D=4×10⁻¹¹ m²/s (Yung, et al. (2010) J. Biomed. Mater. Res. A 95(1):25-32), for the estimation, the diffusion time across 1 mm distance is about 2.5×10⁴ seconds (or 6.9 hours). Therefore, it is reasonable to consider the burst release of the IL-2 is caused by IL-2 detachment from the surface of the toroidal-spiral channel near the TSP opening and diffusion through the collagen biomatrix to the buffer. Despite the fact that the IL-2 molecules were mainly loaded in a layer near the surface of the toroidal-spiral channel, the limiting step of IL-2 release is IL-2 diffusion from the PEG main polymer matrix to the collagen phase. Therefore, the surface of the toroidal-spiral channel coated with IL-2 functioned similar to a catalytic surface for stimulating lymphocyte proliferation and growth.

The biocompatibility of TSPs with collagen filled in the channels was also examined in vivo. The TSPs were subcutaneously implanted in C57BL/6 mice and retrieved two and ten weeks after implantation. Notably, there was no neutrophil infiltration, muscle damage, or vascular change. However, a thin layer of fibrosis (about 70 μm) and a few inflammatory cells were observed. The thickness of the fibrosis changed minimally over time.

### Example 2: Heterogeneous Toroidal Spiral Particles for Islet Encapsulation

Materials and Reagents. Poly (ethylene glycol) diacrylate (PEGDA) MW 700 and glycerol were purchased from Sigma-Aldrich (St. Louis, MO). PEGDA MW 8,000 and MW 20,000 were purchased from Alfa Aesar by Thermo Fisher Scientific (Tewksbury, MA) and PolySciences Inc. (Warrington, PA), respectively. Water used in all experiments was deionized to 18.2 Ωcm (Nanopure II, Barnstead, Dubuque, IA). I-2959 was provided by BASF (Florham park, NJ). Low viscosity, ultrapure sodium alginate (Pronova UP LVG) was purchased from NovaMatrix (Dupont, Norway). Cell culture media, CMRL 1066 (no phenol red, L-glutamine), was purchased from CORNING®. Hanks' balanced salt solutions, HBSS (no Ca⁺⁺, no Mg⁺⁺) and HBSS (Ca⁺⁺, Mg⁺⁺) were purchased from Thermo Fisher Scientific (Whatman, MA). Reagents were purchased from Sigma Aldrich (St. Louis, MO) or Thermo Fisher Scientific (Whatman, MA), unless otherwise noted. Reagents included Tris-buffer saline (TBS) from BIO-RAD (Hercules, CA), insulin solution from bovine pancreas, D-glucose, micro BCA™ protein assay kit, glucose oxidase assay kits, fetal bovine serum (FBS, SAFC Biosciences) and strep/pen (Life Technologies). All materials were purchased at standard grades and used as received.

TSP Formation. TSPs were made through solidification of polymer droplets sedimenting in a miscible solution (FIG. 1B). Briefly, the polymer droplet was protruded through a needle with the tip placed underneath the bulk solution-air interface. Unhindered by the minimal surface tension between the polymer solution and the bulk phase, a toroidal-spiral shape was formed by the viscous forces during injection and sedimentation. Once the desired shape was reached, the polymer was quickly cross-linked using a high intensity UV lamp (˜10 W/cm², Bluewave 75, Dymax; Torrington, CT). The TSPs production rate using a single capillary was approximately one TSP per minute. Larger-scale production can be achieved using a capillary array with designed distance to avoid droplet interaction (Szymusiak, et al. (2012) Soft Matter 8:7556-7559). The polymer solutions contained one type of PEGDA or a mixture of PEGDA of different molecular weights, in addition to glycerol, DI water, ethanol, and photo initiator (I-2959). For larger molecular weight PEGDA (MW 8,000 and 20,000), the solid powder was first dissolved in DI water at 10 wt %. The fluid and flow conditions were designed to achieve the ideal internal structures. In order to make TSPs with channels width ranging from 100 μm-250 μm, the viscosity ratio of the polymer drop and the bulk solution (λ=μ_{drop}/μ_{bulk}) needs to be larger than two (λ>2). Details on the TSPs' fluid dynamic studies and formation have been described (Sharma, et al. (2012) Langmuir 28:729-735). The final polymer solution composition was tailored to achieve instant cross-linking of the desired toroidal-spiral shape (Table 1). After the TSPs were formed, residual glycerol and unreacted PEGDA and I-2959 were removed by dialysis with 30% ethanol for 2 days (with 4 changes), followed by 70% ethanol to sterilize the particles.

TABLE 1

| Name of the | PEGDA | PEGDA | PEGDA | | | |
|---|---|---|---|---|---|---|
| polymer | 700 | 8000 | 20000 | Glycerol | water | I-2959* |
| solution | (wt %) | (wt %) | (wt %) | (w t%) | (wt %) | (wt %) |
| 700-82% | 82 | — | — | 0 | 10 | 0.03 |
| 700-50% | 50 | — | — | 24 | 18 | 0.03 |
| 700-40% | 40 | — | — | 28 | 24 | 0.03 |
| 700-8k-40% | 24 | 16 | — | 34 | 18 | 0.10 |
| 700-20k-32% | 23 | — | 9.7 | 32 | 31 | 0.44 |

*photoinitiator I-2959 is dissolved in ethanol.

Encapsulation of Islets of Langerhans in TSPs. Sterilized TSPs were dehydrated prior to cell encapsulation. Briefly, sterilized particles were placed in water (molecular biology grade) for two hours and rinsed two times. Excess water was carefully aspirated and then the TSPs were dried in a vacuum filter system at room temperature for 15-25 minutes. Human pancreatic islets were isolated according to the conventional biomaterial regulations. Prior to encapsulation, islets were incubated overnight in CMRL 1066 media (L-glutamine, no phenol red) supplemented with 10% FBS and 1% strep/pen at 37° C. and 5% O₂. To prepare for encapsulation, the cells were washed three times with 1 ml, HBSS (no Ca⁺⁺, no Mg⁺⁺) in a tube. In between each wash the cells were gently centrifuged at 800 rpm for 1 minute. After the final wash, an alginate solution was added to the islets pellet and the islets were slowly resuspend. The alginate solution was composed of 2 wt % UPLVG alginate in 300 mM mannitol at pH 7.4.

The islets suspended in the alginate solution were added to the opening of the dehydrated TSPs using a pipette to minimize the stress on islets due to shear, amounting to 6-8 μL per TSP. Capillary force and polymer rehydration drove the islet suspension into the toroidal-spiral channel. The manual process of adding the cell suspension to the TSP took about five seconds per particle for each drop added. In the future, a robotic array may be used for cell loading to reduce the preparation time. Once the cell suspension was added, the particles were allowed to rest for about 15 minutes to allow the cell suspension to be completely entrained into the toroidal-spiral layer of the particles. The cell-loaded particles were then placed in 50 mM CaCl₂) solution (with 150 mM mannitol and 10 mM MOPS, pH 7.4) for three minutes for alginate gelation. Particles loaded with islets were washed two times with HBSS (W/Ca⁺⁺, Mg⁺⁺), placed in CMRL 1066 media supplemented with 10% FBS and 1% strep/pen, and incubated at 37° C. and 5% O₂. Viability of the encapsulated islets was assessed using inclusion/exclusion dyes at different time points over the course of the in vitro incubation. More specifically, the TSPs or the naked islets were washed with HBSS prior to viability tests. The particles or the islets were placed in 1 mL HBSS with 80 μL of 0.45 μM fluorescein diacetate (FDA) in HBSS, which identified live cells, and 10 μL of 14.34 μM propidium iodide (PI) in HBSS, which identified dead cells. Images were taken using fluorescent microscopy, with filters for FDA (excitation/emission 488 nm/520 nm) and PI (excitation/emission 534 nm/617 nm) (Olympus IX70-Japan, equipped with a Tucsen ISH1000 camera, Tucsen Photonics).

Characterization of TSP Mechanical Properties. Mechanical properties of PEGDA hydrogel with various compositions were characterized. The effect of the TSP shape was investigated by comparing TSPs and discs with same material compositions and cross-linking conditions. A polymer disc of consistent volume was prepared by filling a circular TEFLON® mold between two quartz plates followed by UV-light initiated polymer cross-linking. The overall dimensions of the discs (Φ˜3.3 mm and height˜2.5 mm) were similar to those of the TSPs (diameter Φ˜2.3-2.8 mm and height 2.3 mm). Previously to the test, the particles and discs were placed in DI water for 24 hours. Water on the surface was removed with a tissue. The particles or discs were mounted between two plates and compressed until failure at a strain rate of 0.5 s⁻¹ using a Shimadzu EZ-Test Compact Bench Testing Machine (Shimadzu Corporation, Kyoto, Japan).

Polymer Matrix Equilibrium Swelling and Pore Size Calculation. Swelling tests were performed to calculate the mesh size of the polymer matrix, using polymer discs. After fabrication, the polymer discs were washed and left in DI water for 24 hours. The equilibrium swollen weight (W_{swell}) was measured after removing excess water with a tissue. To get the dry weight (W_{dry}) the samples were freeze-dried for about 12 hours until there was no weight change. The mass swelling ratio Q was defined as:

$$Q = \frac{{W}_{swell} - {W}_{dry}}{{W}_{dry}}$$

Q was averaged over 3 measurements for each polymer.

The polymer volume fraction after swelling, ν_{2,s} was calculated as:

$${v}_{2 , s} = \frac{1}{Q \frac{ρ}{{ρ}_{{H}_{2} O}} + 1}$$

where ρ_{H}_{₂}_{O} is the density of the water and ρ is the density of the polymer in its amorphous state at room temperature. For the calculations in this study, the density of water and PEGDA were taken as 1.0 g/cm³ and 1.12 g/cm³, respectively. The average molecular weight between cross-links (Me) can be calculated as follows according to Bray and Merrill equation for hydrogels prepared in the presence of solvent (Peppas & Merrill (1977) J. Appl. Polym. Sci. 21:1763-1770); Bray & Merrill (1973) J. Appl. Polym. Sci. 17:3779-3794),

$$\frac{1}{{M}_{c}} = \frac{2}{{M}_{n}} - \frac{\frac{1}{{V}_{1}} [ ln ( 1 - {v}_{2 , s} ) + {v}_{2 , s} + χ {v}_{2 , s}^{2} ]}{ρ {v}_{2 , r} [ {( \frac{{v}_{2 , s}}{{v}_{2 , r}} )}^{1 / 3} - \frac{1}{2} ( \frac{{v}_{2 , s}}{{v}_{2 , r}} ) ]}$$

where Mₙ is the number average molecular weight of the polymer chains, V₁ is the molar density of water (18 cm³/mol) and ν_{2,r} is the polymer volume fraction in the polymer solution (relaxed state; (Mellott, et al. (2001) Biomaterials 22:929-941), χ is the Flory-Huggins polymer-solvent interaction parameter, which was taken as 0.426 for water and PEG (Merrill, et al. (1993) Biomaterials 14:1117-1126).

The mesh size (ξ) of the hydrogels was related to M_{c} as (Canal & Peppas (1989) J. Biomed. Mater. Res. 23:1183-1193):

$$ξ = l \cdot {n}^{1 / 2} \cdot {C}_{n}^{1 / 2} \cdot {v}_{2 , s}^{- 1 / 3}$$

where, l is the average C—C and C—O bond length in the PEG chain (which is about 1.50 Å), Cₙ=4 is the characteristic ratio for PEG (Merrill, et al. (1993) Biomaterials 14:1117-1126),

$$n = 3 \frac{{M}_{c}}{{M}_{r}}$$

is the number of chemical bonds in the polymer chain between cross-links (Cruise, et al. (1998) Biomaterials 19:1287-1294; Mellott, et al. (2001) Biomaterials 22:929-941; Merrill, et al. (1993) Biomaterials 14:1117-1126) and monomer, and Mᵣ is the molecular weight of each PEG repeat unit (44 g/mol; Merrill, et al. (1993) Biomaterials 14:1117-1126).

In vitro Transport of Glucose and Insulin Through PEG Polymer Films. Thin circular polymer films, with a thickness of 300 μm and a diameter of 2.54 cm, were prepared in a similar way as herein. These films were fully hydrated and then placed in between the two chambers of a custom-made diffusion apparatus. The chambers were sealed with biocompatible O-rings (FDA compliant EPDM). Pseudo one-dimensional diffusion was assumed for the analysis of the diffusion rate, due to the high diameter to thickness ratio (˜63). The bottom chamber was filled with 2-2.5 mL of a solution of bovine insulin (250 μg/mL in tris buffer) or D-glucose (250 μg/mL in Tris buffer). After the polymer film was clapped in between the chambers, 2 mL of tris buffer (lx, pH 7.4) was added to the top chamber.

The diffusion apparatus was placed in an incubator at 37° C. For each measurement, 1 mL solution was collected every 30 or 60 minutes from the top chamber and replaced with fresh buffer of the same volume. The amount of insulin and glucose in the collected solutions were quantified with Micro BCA and glucose oxidase assay kits, respectively, according to the manufacturer's protocol.

The diffusion coefficients of the permeants through the films were calculated based on Fick's law with the assumptions that (1) the diffusion was pseudo one-dimensional, (2) the solutions at the bottom and top chamber were well mixed, and (3) a quasi-steady state implied that the time for the film to equilibrate was much shorter than the diffusion time.

The initial concentration C_{2,0}=0 mg/mL and t₀=0; thus the concentration C₂(t) in the top chamber was fitted with respect to time, t, as given in the following equation to determine the diffusion coefficient (D_{film}; Lee, et al. (2006) Biomaterials 27:1670-1678):

$$- ln ( 1 - \frac{{C}_{2} ( t )}{N / V} ) = \frac{{D}_{film}}{h} ( \frac{t}{τ} )$$

where N is the total solute mass in the system, h is thickness of the film, and V and τ represent geometric parameters of the system given as:

$$V = {V}_{1} + Ah + {V}_{2}$$

$$τ = \frac{( {V}_{1} + Ah / 2 ) ( {V}_{2} + Ah / 2 )}{AV}$$

where A is the area available for diffusion, V₁ and V₂ are the volumes of the solutions in the bottom (donor) and top (receiving) chambers of the diffusion cell. With the initial conditions (N), geometric parameters (A, h, V₁, V₂), and the measurements C₂(t) and t, the diffusion coefficient (Drina) can be fitted by a linear regression C₂(t) as above.

In vitro Glucose-Stimulated Insulin Secretion (GSIS). Encapsulated islets were incubated 24 hours prior to glucose stimulation tests under standard culture conditions. Static glucose-stimulated insulin secretion (GSIS) assay was performed using cell inserts. The un-encapsulated islets (as a control) and islets encapsulated in TSPs were suspended in cell-inserts (12 μm pore size; Millipore Sigma) and incubated in a 24-well plate in Krebs-Ringer buffer (KRB). Each batch (or cell insert) had a total of 100-200 islet equivalent (IEQ) of un-encapsulated or encapsulated islets. The KRB solution was prepared with 119 mM NaCl, 4.7 mM KCl, 25 mM NaHCO₃, 1.2 mM MgSO₄, 1.2 mM KH₂PO₄, 2.5 mM CaCl₂-2H₂O, HEPES (20 mM), and BSA (0.5% wt).

Prior to the assay, the TSPs were rinsed quickly three times with low glucose KRB solution (LG-KRB, 2.8 mM), and then washed three more times with LG-KRB for 10 minutes. The GSIS assay was performed as a sequential incubation of the islets (free or encapsulated in TSPs) in 1 mL glucose-KRB at 37° C. as follows: pre-wash with LG-KRB (2.8 mM) for 30 minutes, pre-incubation in LG-KRB for 60 minutes, then GSIS incubation in LG-KRB for 60 minutes followed by high glucose KRB (HG-KRB, 28 mM) for 60 minutes. The insulin released during the GSIS incubations at LG and HG were the values used to calculate the stimulation index (SI). Insulin concentration was determined by human insulin ELISA kit (ALPCO, NH) as per supplier protocol. Unless otherwise stated, all the measured insulin by ELISA kit was normalized to IEQ.

In vivo Implantation of TSP. TSP in vivo biocompatibility tests were performed in non-immunocompromised C57BL/6 mice. The surgical procedure was briefly as follows. The mice were anesthetized, had their abdomen shaved, and sterilized. An incision was made along the midline of the abdomen and the intraperitoneal lining was exposed. A desired volume of TSPs, sterile and in HBSS, were loaded into a sterile pipette and then implanted into the intraperitoneal cavity through the incision. The incision and the skin were then closed. The surgical animal protocols were regulated and performed at Sparx Therapeutics Inc (Mt. Prospect, IL). TSPs were recovered at 2 weeks and 4 weeks after intraperitoneal transplantation.

In vivo functionality of human islets encapsulated into TSP was assessed using nude mice (Harlan Sprague Dawley Inc). The TSP-encapsulated human islets reached 75-100 IEQ per TSP. Each mouse received 1500-2000 IEQ implantation. The intraperitoneal implantation of TSPs loaded with islets followed a similar procedure as described above. The diabetic mice were induced with 200 mg/kg of streptozotocin (STZ; Sigma) three days prior to the surgical procedure. All surgical animal protocols were approved by UIC animal care committee and performed at CellTrans, Inc.

Blood glucose and body weight were monitored 3 times a week following the transplantation of the TSPs loaded with islets. Mice with non-fasting blood glucose levels below 220 mg/dL were considered normoglycemic. Mice were monitored 28 days post-transplant. At this point the mice were euthanized and the TSPs were retrieved from all mice.

Histology. TSPs retrieved from mice, which were collected at specific time points, were fixed in 10% formalin for 24 hours at room temperature and then transferred to 70% ethanol until being embedded, sectioned, and stained with hematoxylin- and eosin.

Statistical Analysis. Details of sample size (n) are specified. The results were expressed as mean±standard deviation (SD). Significance analysis was performed using one-way analysis of variance (ANOVA) followed by Student (two-tailed) t-tests to compare two sets of data. Results with p<0.05 were considered statistically significant.

Results. The main polymer matrix of the TSPs provides mechanical strength and can provide immune protection from the host immune system. Moreover, the heterogeneous structure of TSPs, in addition to polymer composition, affect their mechanical properties. Stress-strain tests were performed to measure the mechanical properties of the particles and compare them with discs of similar dimensions. In general, polymers with higher polymer concentration and lower molecular weight polymer were less elastic. For the polymer discs, the ultimate strength (stress at break) and compressive modulus increased as the cross-link density increased, which was consistent with the literature (Nii, et al. (2013) Acta Biomaterialia 9:5475-5483). However, for TSPs, the compressive modulus showed no significant change for different polymer compositions and concentrations. The mechanical strength of the particles was strongly influenced by their physical structure and morphology, such as the outer wall thickness, channel width, or the size of the void space in the center, in addition to polymer composition or concentration. Robust mechanical properties of these particles are highly desirable for successful handling and retrievability for possible clinical applications (Darnell, et al. (2013) Biomaterials 34:8042-8048). In this study, the TSPs made of polymer mixtures of PEGDA MW 700-8000 and 700-20,000 have competitive strength compared to robust hydrogel discs (Liu, et al. (2020) Biomaterials 230:119640) and compressive strain compared to regular alginate microcapsules (Bhujbal, et al. (2014) J. Mech. Behav. Biomed. Mater. 37:196-208; Darabbie & Opara (2017) Meth. Molec. Biol. 1479:111-118).

Mesh Size of the Polymer Matrix and Insulin and Glucose Transport. For islets of Langerhans it is critical that the encapsulant allows high permeability of glucose and insulin. Effects of pore size of the hydrogel and of the internal structure on the transport of glucose and insulin were characterized to optimize the design of the TSPs for islet encapsulation and transplantation. Hydrogels of lower polymer concentrations and therefore lower cross-linking densities were chosen for their good mechanical resilience, bigger mesh size, and larger swelling ratio.

Equilibrium swelling tests were conducted on polymer films fabricated of same polymer compositions and cross-linking time as the TSPs. The hydrogel pore size as altered by reducing the total amount of polymer from 50 wt % to 32 wt % and by adding PEGDA of longer sizes of MW 8,000 and 20,000. The films were made of PEGDA MW 700 (50 wt %), a PEGDA mixture of MW 700 and 8,000 (40 wt %), and a PEGDA mixture of MW 700 and 20,000 (32 wt %); referred to hereafter as P700, P700-8k, and P700-20k, respectively (Table 1). The measured average molecular weight (Mc) and mesh size (ξ) between cross-links for P700 in this study was comparable to the projected results described elsewhere for PEGDA MW 575 (Mellott, et al. (2001) Biomaterials 22:929-941) and PEGDA Mw 700 (Majer & Southan (2017) J. Chem. Phys. 146:225101) for hydrogels with polymer concentration ranging from 10 wt % to 30 wt %. As expected, the swelling ratio (Q), Mc, and ξ increased as the cross-linking density decreased. It has been shown that polymer concentration does not have a significant contribution on Mc and ξ of PEGDA hydrogels (Cruise, et al. (1998) Biomaterials 19:1287-1294). Therefore, the increase in Mc and mesh size was attributed mainly to the addition of the longer chain PEGDA.

Diffusion of glucose and insulin through the hydrogel films (thickness of about 300 μm) from a concentrated permeant (insulin or glucose) to a sink buffer solution was evaluated in a diffusion apparatus. Concentration of the permeants (C₂) at the top chamber (sink buffer) of the diffusion apparatus was linearized against collection time (t). The slope of

$$- ln ( 1 - \frac{{C}_{2} ( t )}{N / V} ) vs .$$

t indicated the diffusion coefficient (D_{film}) of the permeant, using geometric parameters of the system. The calculated diffusion coefficients of glucose and insulin through various films were plotted. This analysis indicated that the diffusion of glucose was faster than insulin through the same type of polymer film. This is because glucose is of smaller size, more hydrophilic, and suffers less entanglement within the polymer matrix.

Glucose diffusivity through the films with higher MW (P700-8k and P700-20k) were significantly larger than for P700. Glucose diffusivity through the P700 films (0.67×10⁻⁶ cm²/s) was one order of magnitude slower than in water (D₀=6.7−7×10⁻⁶ cm²/s), despite the mesh size of P700 (˜1 nm) being bigger than the hydrodynamics radius of glucose (˜0.37 nm). Diffusion of glucose through the P700-8k and P700-20k films was three times faster (1.77-1.86×10⁻⁶ cm²/s).

With hydrodynamic radius of insulin being larger than 1 nm (˜1.47-1.6 nm), detectable amounts of diffusion through P700 film were not expected. Thus, insulin diffusion was analyzed for P700-8k and P700-20k films (FIG. 2). Despite the small difference in mesh size calculated for these films, insulin diffusivity through P700-8k was significantly slower than for P700-20k. It has been reported that at neutral buffer conditions, insulin agglomerates in dimers and hexamers. These monomers, dimers and hexamers could be at equilibrium resulting in an aqueous diffusion of 1.14×10⁻⁶ cm²/s.

Notably, the film thicknesses for these studies were 6 to 10 times thicker than TSPs external layer (˜30-50 μm). Therefore, molecular transport across the outer layer of the TSPs could be faster than the conservative static measurements reported above.

Islets Encapsulation and in vitro Viability. TSPs were prepared by a scalable self-assembly process, free of any residual toxin or radiation and ready to use. Within the TSPs high cell density can be achieved while maintaining high cell viability. The islets efficiently occupied the most volume of the particles by distributing along the periphery of the particles and were protected by a thin outer layer of the hydrogel (approximately 30-50 μm thick). There were a small portion of the cells located in the center of the TSPs, which possessed no advantage for the fast mass transport. By counting 15 TSPs, 10.5% of the islets (or 11.0% of IEQ) on average were in the center. The number-weighted average distance of the islets to the outer surface of the TSPs was about 150 μm. As a comparison, for islets homogenously distributed in a spherical bead with 1 mm radius, the theoretical average distance of the islets to the outer surface is 250 μm. The cells located in the center of the particles can be moved to the toroidal-spiral layer along the periphery by adding a small volume of pure alginate solution after adding the cell suspension. However, all the encapsulated islets used in this study were prepared without this additional step to exclude the cells located in the center of the TSPs. On average, the islets in each TSP were about 114 IEQ and the highest loading capacity was up to ˜200 IEQ, based on initial (pre-loading) IEQ count, with minimal loss during cell loading. As a comparison, a similar number of islets can be allocated in one TSP (less than 3 mm in diameter) as in a conventional 8 mm microwell.

The viability of encapsulated islets was monitored at various time points. The TSP-encapsulated islets, with PEGDA 700-8k and 700-20k (both with ˜90 IEQ per TSP), maintained high and similar viability as that of naked islets and alginate capsules. The red fluorescent from the dead cells (with IP dye) was enhanced by adjusting the brightness, compared to the green fluorescence from the live cells. Since an islet is a group of cells, both green and red fluorescence were observed in the same islet. The alginate capsules used as control had similar alginate volume as injected to the TSPs. Similar high cell viability was also achieved with TSPs loaded with non-human primates (NHP) islets (˜160 IEQ per TSP) tested up to 14 days. These individual studies were done per donor and the monitoring time was limited by total number of available islets of the donor.

In these studies, better cell viability was observed for smaller islets, consistent with previous studies showing that smaller islets survived better in normal and reduced oxygen environment with increased insulin secretion (Lehmann, et al. (2007) Diabetes 56:594-603). Accordingly, in some aspects, islets are sorted to obtain cells within the range of 50 to 150 μm.

Islets in vitro Functionality. A GSIS assay was performed to compare the response of the encapsulated islets to glucose stimulation to naked islets. Similar amount of IEQ were encapsulated in the TSPs (about 80 to 100 IEQs per TSP), which were grouped in three sets (n=3), each set with 4-5 TSPs. Compared to naked islets, the stimulation index (SI) values of the encapsulated islets were dependent upon the molecular weight and composition of the polymer matrix. Islets encapsulated within TSPs of lower polymer concentration and higher molecular weight, TSP 700-20k, had similar insulin release and SI as the naked islets (FIG. 3).

TSP Biocompatibility. The TSPs (P700-8k, filled with UPLVG-alginate), retrieved from the IP cavity of immunocompetent C57BL/6 mice showed a minimal immune response. The cells displayed a thin cell lining surrounding the particles, where the largest thickness was found at 2 weeks, ˜40-70 μm. At 4 weeks after implantation, the cell deposition was reduced to less than 40 μm. This reduction was likely due to the surface chemistry of the hydrogel (Sheikh, et al. (2015) Materials 8:5671-5701). In addition, a thicker layer of cell deposition (>70 μm) was noticed at the top of the particles where alginate was exposed. Alginate of a different type, purity, composition, or the type of cross-linking ions (typically Ca²⁺ or Ba²⁺) has been shown to affect the degree of cell overgrowth (Veiseh, et al. (2015) Nature Mater. 14:643-651; Omer, et al. (2005) Transplantation 79:52-58; Paredes-Juarez, et al. (2014) Materials 7:2087-2103; Mørch, et al. (2006) Biomacromolecules 7:1471-1480J), thus leading to foreign body response (FBR). However, more recently, chemical modifications to the alginate backbone (including addition of zwitterionic moieties), have shown a significant reduction or no cell deposition on modified alginate capsules (Bochenek, et al. (2018) Nat. Biomed. Engin. 2:810-821; Vegas, et al. (2016) Nat. Biotechnol. 34:345-352; Liu, et al. (2019) Nat. Commun. 10:1-14). Accordingly, modified alginate can be used to improve the implantation immune response of the TSPs disclosed herein. The formation of some new blood vessels was noticeable at 2 weeks and 4 weeks after implantation. Some of the blood vessels were located around, but mostly at, the top of the TSPs.

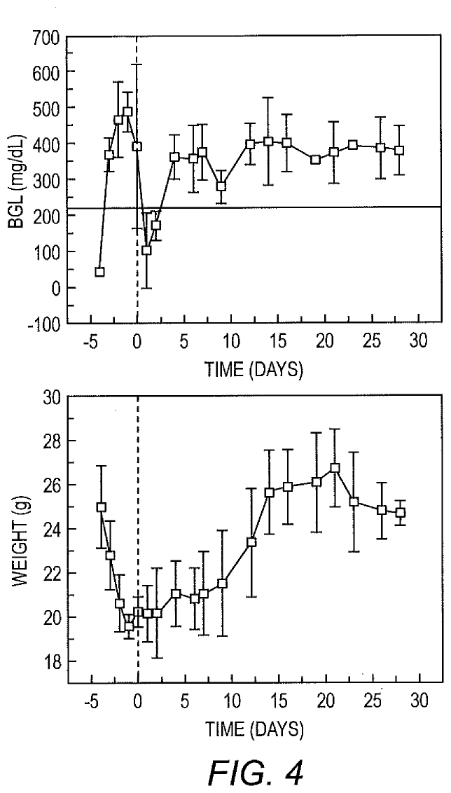

In vivo Functionality. Despite the consistent hyperglycemic levels and stable blood glucose levels (BGL) compared to the normoglycemic cutoff (220 mg/dL, indicated by the line in FIG. 4), mouse body weight increased over the study and the mice showed continuous healthy daily activity until the day of the TSPs retrieval surgery. In this study, each mouse received 1500-2000 IEQ implantation, which was comparable to the dose of the naked islets but relatively low for the encapsulated islets (usually 3000-4000 IEQ). After 4 weeks, the particles were retrieved from mice. The encapsulated islets remaining within the TSP were viable and maintained their morphology in both healthy and STZ-induced diabetic mice (as seen from dithizone staining). Also, the retrieved TSPs showed no damage to their structure, displaying their robustness and supporting the mechanical studies. During retrieval, most of the particles were found attached to tissue within the IP cavity, however those from STZ-diabetic mice were easier to retrieve, TSP retrieval rate was 94% on average. Interestingly, vascular vessels were found around and going into the TSP from the top entrance, similar to the findings previously observed with PEG 700-8k TSPs. While not wishing to be bound by theory, TSP vascularization may enhance oxygen flow to encapsulated islets.

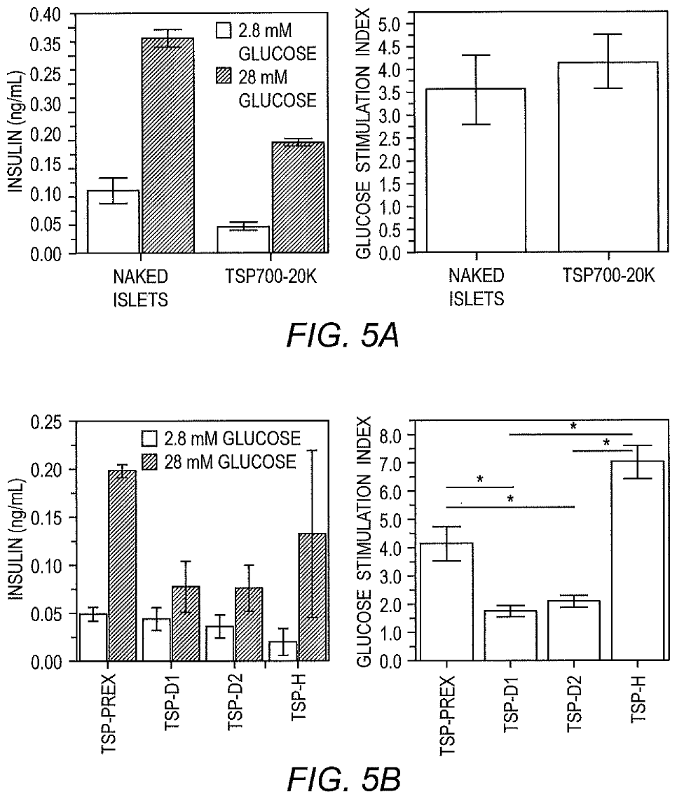

Encapsulated islets demonstrated insulin release and a comparable stimulation index (SI) before transplant compared to naked islets (FIG. 5A). However, glucose responsiveness in diabetic mice was reduced post-transplant (FIG. 5B). This may have been due to the low dose of the TSP-encapsulated islets, for which the islets were over stressed, because the glucose response of the TSP-encapsulated islets retrieved from healthy mice seemed less affected and their glucose SI was not significantly different to encapsulated islets pre-transplant.

Overall, these results demonstrate that encapsulation of islets of Langerhans is achieved with high loading capacity (˜160 IEQ/TSP) and excellent cell viability. TSP-encapsulated islets showed similar glucose-stimulated insulin secretion as naked islets. In addition, biocompatibility of the TSPs on naïve C57BL/6 mice showed minimal inflammatory response after 4-week transplantation into the intraperitoneal (IP) space.

### Example 3: Heterogeneous Toroidal Spiral Particles for CAR-T Cell Encapsulation

Effective deployment of CAR T cell immunotherapy for solid tumors has proven challenging due to CAR T cell fate within tumors. The TSPs of this invention provide a means to address this by enhancing CAR T cell trafficking to the tumor, augmenting the functional persistence of CAR T cells by codelivery of cells together with cytokines and immune checkpoint antibodies, and releasing the cells at the tumor site with a programmable schedule.

To demonstrate the use of TSPs for delivering immune cells, human PBMCs, mouse CD8 T cells, NK cells were encapsulated in TSPs. Accumulative release of a million cells over two weeks from a single TSP was achieved. High viability of the cells released from the particles (>90%) were observed. The proliferation was similar to the cells maintained in media with IL-2. The first four to seven days, the released cells maintained high CD62L^{high}/CD44⁺ central memory (CM) and over time more cells with CD62L^{low}/CD44⁺ effector memory (EM) appeared. Further studies employed CARs derived from a single chain variable fragment (scFv; SS1) that recognizes human mesothelin (MSLN), fused to the T cell receptor-ζ (TCR-ζ) signal transduction domain and an intracellular domain derived from CD28, and 4-1BB (3rd generation, i.e., MSLN-scFv-BEAM-CD28-4-1BB-CD3zeta). Human T cells were activated with CD3/CD28 beads and expanded with 100 IU of IL-2. One day later, cells were redirected with CAR constructs that bind MSLN and trigger TCR and co-stimulatory signals (CD28 and 4-1BB). Cells were expanded for ˜10 days prior to testing their functionality. Notably, the cytolytic activity of expanded MSLN-specific CAR-T cells was validated in cytotoxic assays against ESLN-expressing SKOV3 cells.

To provide controlled release of CAR T cells in the tumor microenvironment, TSPs were prepared with gelatin methacryloyl (GelMA) as the main polymer matrix. Raw gelatin can only form a physical hydrogel at specific concentrations and temperatures, albeit with low mechanical strength. To improve hydrogel stiffness, the gelatin was chemically modified with methacrylic anhydride (MAA). GelMA is synthesized by reacting free amino groups of lysine/hydroxylysine residues in gelatin molecules with MAA in phosphate-buffered saline (PBS) at 50° C. for 3 hours under physiological pH conditions. See, e.g., Van Den Bulcke, et al. (2000) Biomacromolecules 1(1):31-8, and modified methods of the same described by Lee, et al. (2015) RSC Adv. 5:106094. TSPs were prepared with the GelMA by droplet sedimentation in a bulk solution of glycerol and ethanol and subsequent polymer solidification initiated by UV photo crosslinking (see U.S. Pat. Nos. 8,852,645 and 9,974,839).

Stress-strain tests were performed to measure the mechanical properties of the particles. Stress at break from the top and side of the TSPs was approximately 8 kPa and 4 kPa, respectively. Strain at break was approximately 40% and 65% from the top and side, respectively. When implanted subcutaneously in mice, the GelMA TSPs did not exhibit any toxicity. Based upon a 20% loss in weight over a 60-day period in the presence of collagenase (2 U/mL), the GelMA TSPs were biodegradable.

To demonstrate that the GelMA TSPs could maintain cell viability, IL-2 (200 or 400 ng/particle) was encapsulated in the main polymer matrix, close to the internal surface of the toroidal-spiral channel through polymer rehydration, as described in Example 1. Human T cells were subsequently loaded into the vacant internal channel of the TSPs and viability and proliferation were monitored. This analysis indicated that GelMA TSPs containing IL-2 maintained T cell viability for more than 3 weeks. By comparison, TSPs lacking IL-2 were viable for less than 2 weeks.

The rate of IL-2 release from the TSPs into the culture medium was measured for three initial loading amounts of IL-2 (50 ng, 200 ng and 400 ng). For all three groups, only a small amount (approximately 1%) of IL-2 was accumulatively released into the medium in two weeks. Using the method described in Example 1, release of T cells from the GelMA TSPs was also determined in the presence of 200 or 400 ng/TSP IL-2 encapsulated in the main polymer matrix of the TSP. While the release of cells from TSPs containing no IL-2 plateaued after 10 days at 1.2×10⁶ cells, cell release from TSPs containing either amount of IL-2 continued for up to 26 days with similar numbers of cells being released, i.e., approximately 2.6×10⁶ cells released from TSPs containing 200 ng IL-2 and 3.0×10⁶ cells released from TSPs containing 400 ng IL-2.

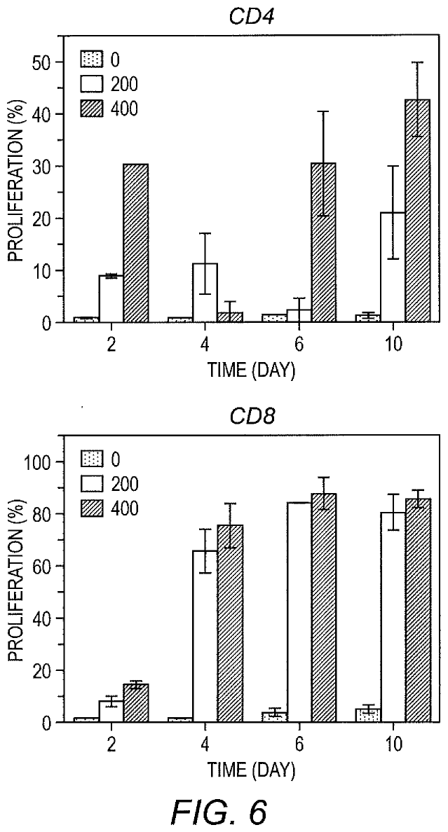

The proliferation of T cells released from the GelMA TSPs was also assessed in vitro. Proliferation of CD4 and CD8 T cells released from TSPs into the culture medium was measured for three initial loading amounts of IL-2 (0 ng, 200 ng and 400 ng). This analysis indicated that CD4 T cells exhibited higher levels of proliferation when released from TSPs containing 400 ng IL-2 whereas CD8 T cells exhibited high levels of proliferation when released from TSPs containing either 200 or 400 ng IL-2 (FIG. 6).

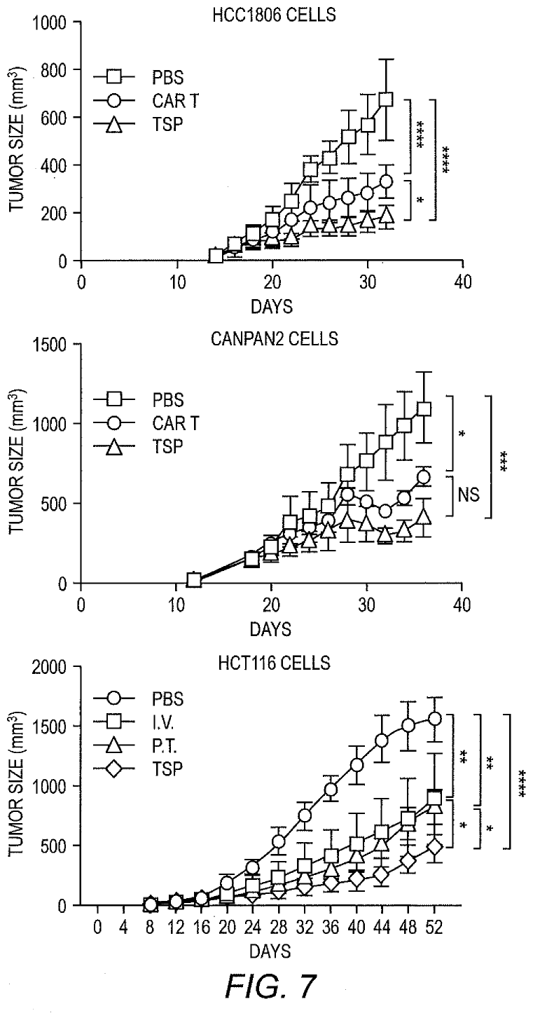

To demonstrate in vivo functionality, anti-mesothelin CAR T cells (anti-MSLN CAR) were tested in NSG mouse (NOD scid gamma mouse) models of triple negative breast cancer (HCC1806 cells), pancreatic cancer (CanPan2 cells), and colorectal carcinoma (HCT116 cells). For this analysis, the human tumor cell lines were engrafted in the NSG mice; anti-MSLN CAR T cells or TSPs containing anti-MSLN CAR T cells were subsequently introduced into the mice (s.c. peritumoral surgical implantation) and tumor size over time was monitored. The results of this analysis (FIG. 7) indicated that TSP loaded with CAR T cells showed a similar or statistically significant reduction in tumor size compared to anti-MSLN CAR T cells alone thereby demonstrating the use of the TSPs of this invention in the treatment of cancer. TSPs loaded with anti-MSLN CAR T cells, one or more supportive cytokines (e.g., IL-2, IL-7, IL-15/15Rα), and optionally a therapeutic agent (e.g., anti-CTLA-4 or anti-PD-1 antibody) provides an effective approach for treating solid tumors, including mesothelioma, breast, lung, ovarian, and pancreatic cancers.

What is claimed is:

1. An in situ cell bioreactor and delivery system comprising toroidal-spiral particles, the toroidal-spiral particles comprising a biocompatible polymer matrix forming a channel and a compartment operatively connected to said channel, wherein said channel encapsulates cells suspended in a scaffold material that modulates activity or release of the cells from the toroidal-spiral particles and said compartment optionally comprises one or more active agents.

2. The system of claim 1 wherein the biocompatible polymer matrix comprises polyethylene glycol diacrylate, polyethylene glycol methacrylate, gelatin methacryloyl, methacrylated hyaluronic acid, methacrylated chitosan, methacrylated chondroitin sulfate, methacrylated glycol chitosan, methacrylated alginate, methacrylated dextran, methacrylated gellan gum, methacrylated poly(ε-caprolactone), dimethacrylate poly-D,L-lactide, dextran hydroxyl ethyl methacrylate, dextran mono(2-acryloyloxyethyl) succinate, polyglycerol-co-sebacate acrylate, α,ω-diacrylate polyethylene carbonate, polytrimethylene carbonate methacrylate, polyglycerol-co-sebacate-cinnamate, polyethylene glycol methyl ether methylacrylate, ethylene glycol dimethacrylate, polypropylene glycol methacrylate, or combinations or copolymers thereof.

3. The system of claim 1, wherein the cells comprise immune cells, chimeric antigen receptor T cells, T cells engineered to express modified T cell receptors, stem cells, or islets of Langerhans.

4. The system of claim 1, wherein the scaffold material comprises collagen, alginate, chitosan, fibrin, keratin, polyacrylamide, polyethylene glycol, hyaluronic acid, or a combination thereof or copolymer thereof.

5. The system of claim 1, wherein the one or more active agents comprise immune checkpoint antibodies, therapeutic proteins or peptides, cancer chemotherapeutic drugs, immunoregulators, cytokines, vitamins, nutrients, diagnostic contrast agents, quantum dots, polymeric nanoparticles, or lipid nanoparticles.

6. The system of claim 1, wherein the one or more active agents reside on a surface of the compartment, in the compartment, in the channel, or a combination thereof.

7. The system of claim 1, wherein the cells comprise islets of Langerhans and the scaffold material comprises alginate.

8. The system of claim 1, wherein the cells comprise immune cells or chimeric antigen receptor T cells, the scaffold material comprises collagen, and the one or more active agents comprise IL-2.

9. A method for producing the in situ cell bioreactor and delivery system of claim 1 comprising
(a) preparing toroidal-spiral particles comprising a biocompatible polymer matrix forming a channel and a compartment operatively connected to said channel;
(b) dehydrating the toroidal-spiral particles; and
(c) introducing into the channel of the toroidal-spiral particles cells suspended in a scaffold material that modulates activity or release of the cells from the toroidal-spiral particles thereby producing the cell delivery system.

10. The method of claim 9, further comprising introducing one or more active agents into the compartment of the toroidal-spiral particles prior to step (c).

11. A kit for preparing an in situ cell bioreactor and delivery system comprising
(a) dehydrated toroidal-spiral particles comprising a biocompatible polymer matrix forming a channel and a compartment operatively connected to said channel, wherein one or more active agents reside on a surface of the compartment, in the compartment, in the channel, or a combination thereof; and
(b) a container comprising a scaffold material that modulates activity or release of the cells from the toroidal-spiral particles.

12. The kit of claim 11, wherein the biocompatible polymer matrix comprises polyethylene glycol diacrylate, polyethylene glycol methacrylate, gelatin methacryloyl, methacrylated hyaluronic acid, methacrylated chitosan, methacrylated chondroitin sulfate, methacrylated glycol chitosan, methacrylated alginate, methacrylated dextran, methacrylated gellan gum, methacrylated poly(ε-caprolactone), dimethacrylate poly-D,L-lactide, dextran hydroxyl ethyl methacrylate, dextran mono(2-acryloyloxyethyl) succinate, polyglycerol-co-sebacate acrylate, α,ω-diacrylate polyethylene carbonate, polytrimethylene carbonate methacrylate, polyglycerol-co-sebacate-cinnamate, polyethylene glycol methyl ether methylacrylate, ethylene glycol dimethacrylate, polypropylene glycol methacrylate, or combinations or copolymers thereof.

13. The kit of claim 11, wherein the scaffold material comprises collagen, alginate, chitosan, fibrin, keratin, polyacrylamide, polyethylene glycol, hyaluronic acid, or a combination thereof or copolymer thereof.

14. The kit of claim 11, wherein the one or more active agents comprise immune checkpoint antibodies, therapeutic proteins or peptides, cancer chemotherapeutic drugs, immunoregulators, cytokines, vitamins, nutrients, diagnostic contrast agents, quantum dots, polymeric nanoparticles, or lipid nanoparticles.

15. A method for delivering cells to a subject comprising administering to a subject in need of treatment with a cell therapy an effective amount of the in situ cell bioreactor and delivery system of claim 1.

16. The method of claim 15, wherein the subject has cancer, the cells comprise chimeric antigen receptor T cells, the one or more active agents comprise IL-2 and optionally a checkpoint antibody.

17. The method of claim 15, wherein the subject has diabetes, the cells comprise islets of Langerhans, and the scaffold material comprises alginate.
